(19) United States
(12) Patent Application Publication
(10) Pub. No.: US 2021/0206802 A1
(43) Pub. Date: Jul. 8, 2021

(54) MONOTHIOETHER CROSSLINKERS IN POLYMERS AND APPLICATIONS THEREOF

(71) Applicants: Zymergen Inc., Emeryville, CA (US); The Regents of the University of California, Oakland, CA (US)

(72) Inventors: John McNamara, El Sobrante, CA (US); Nicole G. Ricapito, Greenwhich, NY (US); Timothy J. Deming, Los Angeles, CA (US); Eric G. Gharakhanian, San Leandro, CA (US)

(21) Appl. No.: 17/250,094

(22) PCT Filed: May 30, 2019
(86) PCT No.: PCT/US2019/034704

§ 371 Date: Nov. 24, 2020

Related U.S. Application Data
(60) Provisional application No. 62/678,244, filed on May 30, 2018.

(51) Int. Cl. C07K 5/02; B01J 20/24; B01J 20/28; A61K 47/42; A61K 9/06; A61L 27/22; A61L 27/52; C07K 7/02

(52) U.S. Cl. CPC C07K 5/0215; B01J 20/24; B01J 20/28047; C07K 7/02; A61K 9/06; A61L 27/22; A61L 27/52; A61K 47/42

(57) ABSTRACT

A polypeptide comprises amino acids in an amino acid sequence, wherein a portion of the amino acids are covalently linked via a monothioether bridge. In one embodiment, the portion includes amino acids selected from (I) herein n is 0, 1, or 2 and m is 1 or 2. In another embodiments, a hydrogel can include such an amino acid as a crosslinker.

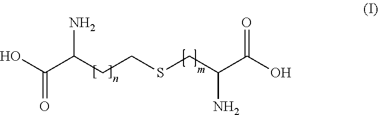

### CROSS-REFERENCE TO RELATED APPLICATIONS

This application claims priority to and benefit of U.S. Ser. No. 62/678,244, filed on May 30, 2018, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### FIELD OF THE DISCLOSURE

The present invention relates generally to the area of polymers and more specifically, to redox-stable polymers and to the area of hydrogels.

### BACKGROUND

Supramolecular structures are held together by intermolecular interactions that are responsible for the organization of polymeric systems. The non-covalent, intermolecular forces which are required for the assembly of the defined supramolecular structures are mainly electrostatic interaction, hydrogen bonding, van der Waals force, etc. Supramolecular chemistry or biology gathers a vast body of two or three dimensional complex structures and entities formed by association of chemical or biological species. These associations are governed by the principles of molecular complementarity or molecular recognition and self-assembly. The knowledge of the rules of intermolecular association can be used to design polymeric assemblies in form of membranes, films, layers, micelles, tubules, gels for a variety of biomedical or technological applications (J.-M. Lehn, Science, 295, 2400-2403, 2002). Peptides have been used for the fabrication of supramolecular structures through molecular self-assembly (S. Zhang, Nature Biotechnology, 21, 1171-1178, 2003). Peptides are for instance able to assemble into nanotubes (U.S. Pat. No. 7,179,784) or into supramolecular hydrogels consisting of three dimensional scaffolds with a large amount of around 98-99% immobilized water or aqueous solution. The peptide-based biomaterials are powerful tools for potential applications in biotechnology, medicine and even technical applications. Depending on the individual properties these peptide-based hydrogels are thought to serve in the development of new materials for tissue engineering, regenerative medicine, as drug and vaccine delivery vehicles or as peptide chips for pharmaceutical research and diagnosis (E. Place et al., Nature Materials, 8, 457-470, 2009). There is also a strong interest to use peptide-based self-assembled biomaterial such as gels for the development of molecular electronic devices (A. R. Hirst et al. Angew. Chem. Int. Ed., 47, 8002-8018, 2008).

A variety of “smart peptide hydrogels” have been generated that react on external manipulations such as temperature, pH, mechanical influences or other stimuli with a dynamic behavior of swelling, shrinking or decomposing. Nevertheless, these biomaterials are still not “advanced” enough to mimic the biological variability of natural tissues as for example the extracellular matrix (ECM) or cartilage tissue or others. The challenge for a meaningful use of peptide hydrogels is to mimic the replacing natural tissues not only as “space filler” or mechanical scaffold, but to understand and cope with the biochemical signals and physiological requirements that keep the containing cells in the right place and under “in vivo” conditions (R. Fairman and K. Akerfeldt, Current Opinion in Structural Biology, 15, 453-463, 2005). Much effort has been undertaken to understand and control the relationship between peptide sequence and structure for a rational design of suitable hydrogels. In general hydrogels contain macroscopic structures such as fibers that entangle and form meshes. Most of the peptide-based hydrogels utilize as their building blocks β-pleated sheets which assemble to fibers. Later it was shown that it is possible to design hydrogelating self-assembling fibers purely from α-helices. Besides β-sheet structure-based materials (S. Zhang et al., PNAS, 90. 3334-3338. 1993: A. Aggeli et al., Nature, 386, 259-262, 1997, etc.) a variety of α-helical hydrogels has been developed (W. A. Petka et al., Science, 281, 389-392, 1998; C. Wang et al., Nature, 397, 417-420, 1999; C. Gribbon et al., Biochemistry, 47, 10365-10371, 2008; E. Banwell et al., Nature Materials, 8, 596-600, 2009, etc.).

Nevertheless, the currently known peptide hydrogels are in most of the cases associated with low rigidity, sometimes unfavorable physiological properties and/or complexity and the requirement of substantial processing thereof which leads to high production costs.

### SUMMARY OF THE INVENTION

In a first aspect, a polypeptide comprises amino acids in an amino acid sequence, wherein a portion of the amino acids are covalently linked via a monothioether bridge. In one embodiment, the portion includes amino acids selected from

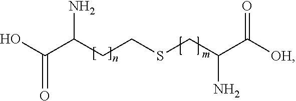

wherein n is 0, 1, or 2 and m is 1 or 2.

In a second aspect, a hydrogel comprises a polymer having a crosslinker. The crosslinker can include the following structure:

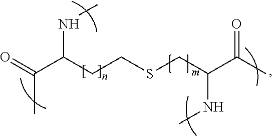

wherein n is 0, 1, or 2 and m is 1 or 2.

In a third aspect, a polypeptide comprises amino acids in an amino acid sequence. A portion of the amino acid sequence can include an amino acid select from

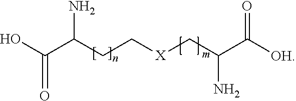

In the structure, n can be 0, 1, or 2 and m can be 1 or 2. X can be any bridging moiety. In one embodiment, X can be selected from a single bond, CH₂, CH(OH), C(O), NH, O, S(O), S(O)₂, or Se.

In a fourth aspect, a hydrogel comprising a polymer having a crosslinker comprising a structure:

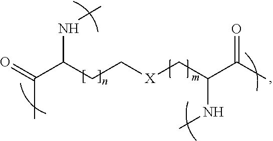

wherein n is 0, 1, or 2 and m is 1 or 2. X can be any bridging moiety. In one embodiment, X can be selected from a single bond, CH₂, CH(OH), C(O), NH, O, S(O), S(O)₂, or Se.

### DETAILED DESCRIPTION

In an embodiment, a polypeptide comprises amino acids in an amino acid sequence, wherein a portion of the amino acids are covalently linked via a monothioether bridge. In one embodiment, the portion can be at least 0.001%, at least 0.005%, at least 0.01%, at least 0.03%, at least 0.05%, at least 0.07%, at least 0.09%, at least 0.1%, at least 0.15%, at least 0.2%, at least 0.25%, at least 0.3%, at least 0.35%, at least 0.4%, at least 0.45%, at least 0.5%, or at least 0.6% of the amino acids are covalently linked via a monothioether bridge. In yet another embodiment, the portion can be not greater than 99%, not greater than 98%, not greater than 95%, not greater than 90%, not greater than 80%, not greater than 70%, not greater than 60%, not greater than 50%, not greater than 40%, not greater than 30%, not greater than 25%, not greater than 20%, not greater than 15%, not greater than 10%, not greater than 9%, not greater than 8%, not greater than 7.5%, not greater than 7%, not greater than 6.5%, not greater than 6%, not greater than 5.5%, not greater than 5%, not greater than 4.8%, not greater than 4.6%, not greater than 4.4%, not greater than 4.2%, not greater than 4%, not greater than 3.8%, not greater than 3.6%, not greater than 3.4%, not greater than 3.2%, not greater than 3%, not greater than 2.8%, not greater than 2.6%, not greater than 2.4%, not greater than 2.2%, not greater than 2%, not greater than 1.8%, or not greater than 1.6% of the amino acid sequence. In one particular embodiment, the portion ranges between 0.001% and 80%, such as between 0.005% and 60%, between 0.01% and 50%, or between 0.02% and 40% of the amino acid sequence.

A portion is understood as the number of amide bonds that a particular amino acid or set of amino acids contributes to the entire polypeptide. For example, the amino acid cysteine forms only one amide bond within an amino acid sequence, thus in a polypeptide of 100 amino acids with only one cysteine, the cysteine portion is 1% of the amino acid sequence. Conversely, cystine, which is the oxidized dimer of cysteine comprising a disulfide bridge forms two amide bonds in a polypeptide, therefore each cystine contributes double to the amino acid sequence. It follows that an amino acid sequence of 99 amino acids, one of which is cystine forms a polypeptide with 100 amide bonds, 2 of which are contributed by cystine and therefore the cystine portion is 2%. As another example, if a polypeptide comprises of 98 standard amino acids which includes 2 cystines, one cysteine, and one methionine, then the sulfur-containing amino acid portion of that polypeptide is 6% because 4 amide bonds are contributed by the cystines, and one each for cysteine and methionine, resulting in 6 amide bonds of a total of 100 amide bonds in the sequence (96 amino acids forming single amide bonds, 2 cystines forming two amide bonds, totaling 100 amide bonds).

In another embodiment, the portion includes an amino acid selected from the group consisting of:

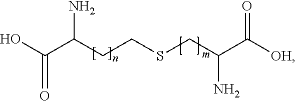

wherein n and m, independently, can be 0, 1, 2, or 3. In one embodiment, n+m is greater than 0, greater than 1, or greater than 2. In one embodiment, the amino acid can be selected from the group consisting of

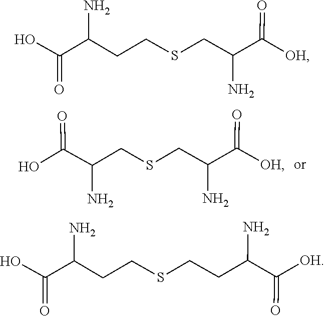

In one embodiment, the amino acid is selected from cystathionine, lanthionine, homolanthionine, or any combination thereof. In one further embodiment, the amino acid is selected from

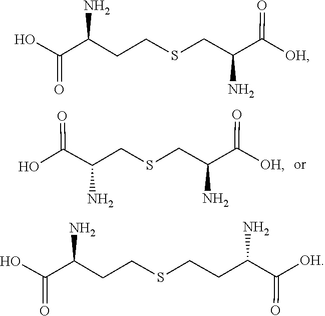

In another embodiment, the amino acids not included in the monothioether-linked portion of the polypeptide are selected from a subset. The subset can include not more than 10 amino acids, such as not more than 9 amino acids, not more than 8 amino acids, not more than 7 amino acids, not more than 6 amino acids, not more than 5 amino acids, not more than 4 amino acids, not more than 3 amino acids, or not more than 2 amino acids. Those amino acids can be selected from the group consisting of alanine, arginine, asparagine, aspartic acid, cysteine, homocysteine, 2,4-diaminobutyric acid, glutamic acid, homoglutamic acid, glutamine, homoglutamine, glycine, histidine, isoleucine, leucine, lysine, methionine, phenylalanine, omithine, proline, serine, homoserine, threonine, tryptophan, tyrosine, and valine. In one embodiment, those amino acids can be selected from a more narrow subset of amino acids, such as all acids with hydrophilic residues, all amino acids with basic residues, all amino acids with aromatic residues, or any combination thereof.

In yet another embodiment, the polypeptide can further include a cysteine portion. The cysteine portion includes the sulfur containing residues in various redox stages. For example, the cysteine portion can include residues with free thiol groups (—SH) or its oxidized variants, such as disulfides (—S—S—), trisulfides (—S—S—S—), or any higher analog oligosulfides. The cysteine portion can be at least 0.01%, such as at least 0.02%, at least 0.04%, at least 0.06%, at least 0.08%, at least 0.1%, at least 0.15%, at least 0.2%, at least 0.25%, at least 0.3%, at least 0.35%, at least 0.4%, at least 0.45%, at least 0.5%, at least 0.55%, at least 0.6%, at least 0.65%, at least 0.7%, at least 0.75%, or at least 0.8% of the amino acid sequence. In one particular embodiment, the cysteine portion includes or consists essentially of cysteine, cystine, homocysteine, homocystine, and/or any combination thereof.

In yet another embodiment, the polypeptide includes a ratio of the monothioether bridges and the cysteine portion. The ratio is calculated as the percentage of monothioether containing amino acids of the polypeptide sequence divided by the percentage of cysteine-type amino acids in the polypeptide sequence, wherein cysteine-type amino acids includes cysteine, cystine, homocysteine, homocysteine, and their oligosulfur homologs. In one embodiment, the number of monothioether bridges is equal or less than the number of cysteine-type amino acids. It follows the ratio is not greater than 1, such as not greater than 0.95, not greater than 0.9, not greater than 0.85, not greater than 0.8, not greater than 0.75, not greater than 0.7, not greater than 0.65, not greater than 0.6, not greater than 0.55, not greater than 0.5, or not greater than 0.45.

In yet one other embodiment, the number of monothioether bridges is higher than the number of cysteine-type amino acids. It follows the ratio is greater than 1, such as greater than 1.1, greater than 1.2, greater than 1.3, greater than 1.4, greater than 1.5, greater than 1.6, greater than 1.8, greater than 2, greater than 2.4, greater than 2.8, greater than 3.2, greater than 3.6, greater than 4, greater than 4.5, greater than 5, greater than 5.5, greater than 6, greater than 7, greater than 8, greater than 10, or greater than 15

In one further embodiment, the polypeptide can comprise the structure:

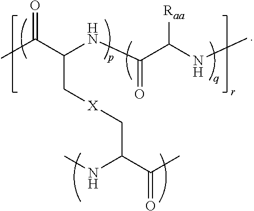

In the foregoing structure, X, for each occurrence, can be independently selected from S, S—CH₂, CH₂—S, S—S, CH₂—S—S—CH₂, or CH₂—S—CH₂. Rₐₐ is an amino acid residue. Rₐₐ can be any of the foregoing mentioned amino acids. Moreover, Rₐₐ, for each occurrence can be a different amino acid. Yet, in another embodiment, Rₐₐ can be the same amino acid for each occurrence. For an embodiment, when Rₐₐ is the same amino acid for a section of the polypeptide, polypeptides can be generated that allow for fine-tuning particular macroscopic properties. For example, in accordance with the types of amino acids selected for the polypeptide and their percentage in the sequence, the polypeptide can result in stimuli-responsive gels and hydrogels. Segments within such gels can exhibit either a lower critical solution temperature (LCST) of an upper critical solution temperature (UCST) wherein the polymer solution or gel exhibits two solubility boundaries.

Still referring to the foregoing structure, the parameter p and q are integers. They can be independently selected from 1 through 200, such as 1 through 150, 1 through 100, or 1 through 50. A fraction p/q can be ≤1, such as ≤0.95, ≤0.90, ≤0.85, ≤0.80, ≤0.75, ≤0.70, ≤0.65, ≤0.6, ≤0.55, ≤0.5, ≤0.45, ≤0.4, ≤0.35, ≤0.30, ≤0.25, ≤0.20, ≤0.15, ≤0.10, ≤0.08, ≤0.06, ≤0.05, ≤0.04, ≤0.03, ≤5 0.02, ≤0.01, ≤0.005, or ≤0.001. The parameter r is an integer not less than 20, not less than 30, not less than 40, not less than 50, or not less than 80.

In one further embodiment, the hydrogel can include a section. The selection can be selected from a polypeptide, a polysaccharide, a polyethylene glycol, a poly vinyl alcohol, a polyacrylate, a polyurethane, a polyamine, or any combination thereof.

In one further aspect, a polypeptide includes amino acids in an amino acid sequence. A portion of the amino acid sequence includes an amino acid selected from

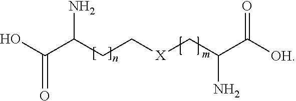

In one embodiment, n is 0, 1, or 2. In another embodiment, m is 1 or 2. In one embodiment, m+n≥1, such as m+n≥2, or m+n≥3. In one embodiment, X can be selected from a single bond, CH₂, CH(OH), C(O), NH, O, S(O), S(O)₂, or Se.

In one embodiment, the portion can at least 0.001% of the amino acid sequence, such as at least 0.005%, at least 0.01%, at least 0.03%, at least 0.05%, at least 0.07%, at least 0.09%, at least 0.1%, at least 0.15%, at least 0.2%, at least 0.25%, at least 0.3%, at least 0.35%, at least 0.4%, at least 0.45%, at least 0.5%, or at least 0.6% of the amino acid sequence. In yet another embodiment, the portion can be not greater than 95% of the amino acid sequence, such as not greater than 90%, not greater than 85%, not greater than 80%, not greater than 75%, not greater than 70%, not greater than 65%, not greater than 60%, not greater than 55%, not greater than 50%, not greater than 45%, not greater than 40%, not greater than 35%, not greater than 30%, not greater than 28%, not greater than 26%, not greater than 24%, not greater than 22%, not greater than 20%, not greater than 18%, not greater than 16%, not greater than 14%, not greater than 12%, not greater than 10%, not greater than 9%, not greater than 8%, not greater than 7%, not greater than 6%, not greater than 5%, not greater than 4%, not greater than 3%, or not greater than 2% of the amino acid sequence.

In another embodiment, the amino acids that are not included in the portion are selected from a subset. The subset can include not more than 10 amino acids, not more than 9 amino acids, not more than 8 amino acids, not more than 7 amino acids, not more than 6 amino acids, not more than 5 amino acids, not more than 4 amino acids, not more than 3 amino acids, or not more than 2 amino acids. The amino acids in the subset can be selected from the group consisting of alanine, arginine, asparagine, aspartic acid, cysteine, homocysteine, 2,4-diaminobutyric acid, glutamic acid, homoglutamic acid, glutamine, homoglutamine, glycine, histidine, isoleucine, leucine, lysine, methionine, phenylalanine, omithine, proline, serine, homoserine, threonine, tryptophan, tyrosine, and valine.

In yet another embodiment, a section of amino acids that is not included in the portion is not greater than 99%, not greater than 98%, not greater than 95%, not greater than 90%, not greater than 80%, not greater than 70%, not greater than 60%, not greater than 50%, not greater than 40%, not greater than 30%, not greater than 25%, not greater than 20%, not greater than 15%, not greater than 10%, not greater than 9%, not greater than 8%, not greater than 7.5%, not greater than 7%, not greater than 6.5%, not greater than 6%, not greater than 5.5%, not greater than 5%, not greater than 4.8%, not greater than 4.6%, not greater than 4.4%, not greater than 4.2%, not greater than 4%, not greater than 3.8%, not greater than 3.6%, not greater than 3.4%, not greater than 3.2%, not greater than 3%, not greater than 2.8%, not greater than 2.6%, not greater than 2.4%, not greater than 2.2%, not greater than 2%, not greater than 1.8%, or not greater than 1.6% of the amino acid sequence.

In one further aspect, a hydrogel comprises a polymer having a crosslinker. The crosslinker can include a structure:

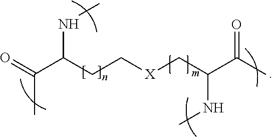

In one embodiment, n is 0, 1, or 2. In another embodiment, m is 1 or 2. In one embodiment, m+n≥1, such as m+n≥2, or m+n≥3. In yet another embodiment, X can be selected from a single bond, CH₂, CH(OH), C(O), NH, O, S(O), S(O)₂, or Se.

In further embodiments, the hydrogel has at least one property selected from the group consisting of:
(i) the hydrogel maintains gelation in the presence of a reduction agent selected from a mercapto compound or dithiothreitol;
(ii) the hydrogel is an element of a pharmaceutical formulation adapted to release an active ingredient;
(iii) the hydrogel is an element of an engineered tissue for biological tissue replacement;
(iv) the hydrogel is adapted to absorb pollutants from a liquid;
(v) the hydrogel is an element of an electrode adapted to measure one or more carbohydrates present in organic and biological fluids and tissues;
(vi) the hydrogel is selfhealing;
(vii) the hydrogel has a peel strength according to ASTM F2256 (2015) of at least 0.1 N, at least 0.2 N, at least 0.4 N, at least 0.6 N, at least 0.8 N, at least 1.0 N, at least 1.5 N, at least 2 N, at least 4 N, at least 6 N, at least 8 N, or at least 10 N; and
(viii) any combination of (i), (ii), (iii), (iv), (v), (vi), and (vii).

With respect to pollutants, the hydrogel can absorb the following pollutants: pollutants: acenaphthene, acrolein, acrylonitrile, benzene, benzidine, carbon tetrachloride, chlorobenzene, 1,2,4-trichlorobenzene, hexachlorobenzene, 1,2-dichloroethane, 1,1,1-trichloreothane, hexachloroethane, 1,1-dichloroethane, 1,1,2-trichloroethane, 1,1,2,2-tetrachloroethane, chloroethane, bis(2-chloroethyl) ether, 2-chloroethyl vinyl ethers, 2-chloronaphthalene, 2,4,6-trichlorophenol, parachlorometa cresol, chloroform, 2-chlorophenol, 1,2-dichlorobenzene, 1,3-dichlorobenzene, 1,4-dichlorobenzene, 3,3-dichlorobenzidine, 1,1-dichloroethylene, 1,2-trans-dichloroethylene, 2,4-dichlorophenol, 1,2-dichloropropane, 1,3-dichloropropylene, 2,4-dimethylphenol, 2,4-dinitrotoluene, 2,6-dinitrotoluene, 1,2-diphenylhydrazine, ethylbenzene, fluoranthene, 4-chlorophenyl phenyl ether, 4-bromophenyl phenyl ether, bis(2-chloroisopropyl) ether, bis(2-chloroethoxy) methane, methylene chloride, methyl chloride, methyl bromide, bromoform, dichlorobromomethane, chlorodibromomethane, hexachlorobutadiene, hexachlorocyclopentadiene, isophorone, naphthalene, nitrobenzene, 2-nitrophenol, 4-nitrophenol, 2,4-dinitrophenol, 4,6-dinitro-o-cresol, n-nitrosodimethylamine, n-nitrosodiphenylamine, n-nitrosodi-n-propylamine, pentachlorophenol, phenol, bis(2-ethylhexyl) phthalate, butyl benzyl phthalate, di-n-butyl phthalate, di-n-octyl phthalate, diethyl phthalate, dimethyl phthalate, benzo(a) anthracene, benzo(a) pyrene, benzo(b) fluoranthene, benzo(k) fluoranthene, chrysene, acenaphthylene, anthracene, benzo(ghi) perylene, fluorene, phenanthrene, dibenzo(h) anthracene, indeno (1,2,3-cd) pyrene, pyrene, tetrachloroethylene, toluene, trichloroethylene, vinyl chloride, aldrin, dieldrin, chlordane, 4,4-DDT, 4,4-DDE, 4,4-DDD, alpha-endosulfan, beta-endosulfan, endosulfan suffate, endrin, endrin aldehyde, heptachlor, heptachlor epoxide, alpha-BHC, beta-BHC, gamma-BHC, delta-BHC, PCB-1242 (arochlor 1242), PCB-1254 (arochlor 1254), PCB-1221 (arochlor 1221), PCB-1232 (arochlor 1232), PCB-1248 (arochlor 1248), PCB-1260 (arochlor 1260), PCB-1016 (arochlor 1016), toxaphene, antimony, arsenic, asbestos, beryllium, cadmium, chromium, copper, cyanide, lead, mercury, nickel, selenium, silver, thallium, zinc, or 2,3,7,8-TCDD.

In addressing the aforementioned peel strength, in some embodiments, the sequestered curing agents used herein may permit formation of an adhesive material that is adherent for a sufficient period to permit the tissue to repair themselves but is not necessarily designed to act as a permanent adhesive. For example, the sequestered curing agent and other materials can be selected to provide a bioadhesive that serves as a temporary interface while the body's natural healing mechanisms repair the tissue damage and reestablish the bonding of two separated tissue planes. The separation of tissue layers is commonly encountered in medicine. The development of seromas, which is an accumulation of fluid between tissue layers, is a critical problem and one example of a possible use for the adhesives described herein is to avoid formation of seromas. In some instances, the adhesive may provide adherence between the tissues in situ for at least 7 days, more particularly, at least 10 days or at least 14 days. In certain embodiments, the adhesive strength of the adhesive materials may be at least 50 N as tested by ASTM F2255 dated 2003, more particularly at least 60 N as tested by ASTM F2255 dated 2003 or at least 70 N as tested by ASTM F2255 dated 2003. In certain instances, the T-peel strength as tested by ASTM F2256 dated 2005 may be at least 0.20 N, for example, at least 0.50 N as tested by ASTM F2256 dated 2005 or at least 0.70 N as tested by ASTM F2256 dated 2005. During the tissue repair process, the bioadhesive may be resorbed, degraded, etc. or may be used as a makeshift framework to permit cell ingrowth and/or stabilization during the tissue repair process.

In another embodiment, the hydrogel can further include a section. The section can be selected from the group consisting of a polypeptide, a polysaccharide, a polyethylene glycol, a poly vinyl alcohol, a polyacrylate, a polyurethane, a polyamine, or any combination thereof.

Note that not all of the activities described above in the general description or the examples are required, that a portion of a specific activity may not be required, and that one or more further activities may be performed in addition to those described. Still further, the order in which activities are listed are not necessarily the order in which they are performed.

It is well known in the art that synthetic polypeptide network materials comprised of disulfide amino acid cross-linker, like cystine and homocystine, are susceptible to reductive cleavage and degradation. Common chemical reducing agents, such as, mercaptoethanol, dithiothreitol (DTT), and tris(2-carboxyethyl)phosphine (TCEP) are known to reductively cleave the disulfide bond thereby disrupting and reducing the material's structural integrity. In addition to chemically-induced reduction, the disulfide bonds can be cleaved using cathodically-induced electro-chemical reduction. By placing the disulfide cross-linked material in an electrochemical cell, it is known that the disulfide bonds are cleaved at the cell cathode. It is possible to recover the lost structural integrity. Using chemical oxidizing agents and anodic electro-chemical oxidation the disulfide bonds can be reformed, thereby rebuilding the material's cross-linked network.

The stability of cross-linked synthetic polypeptide network materials can be improved or be rendered inert to the aforementioned oxidation and reduction (REDOX) when employing crosslinker that do not include disulfide bridges, e.g., monosulfide amino acids as the network crosslinker. Monosulfides and monosulfide cross-linked networks remain intact and resistant to cleavage under the same reduction conditions used to cleave disulfides. In other words, stable synthetic polypeptide network materials are prepared using one or more monosulfide amino acids and amino acids. Therefore, if a polypeptide is included in a hydrogel and the cross-linker include such monosulfide crosslinkers or crosslinkers that are resistant to reduction and oxidation, such hydrogels do maintain their gelation state upon treatment with a reducing agent as described above.

In one embodiment, the monosulfide amino acid cross-linkers of the stable synthetic polypeptide network materials can be comprised, e.g., of one or more cystathionine, lanthionine, and homolanthionine amino acids. Said monosulfide amino acid cross-linkers can also originate from fermentation using gram negative bacteria, gram positive bacteria, yeast, and fungi. In embodiments, the stable polypeptide network materials can be used to form stable gels and hydrogels, wherein the cross-linked network of said gel and hydrogel maintains it structural integrity under reductive and oxidative environments.

Preparation of the polymers can, for example, according to the following scheme:

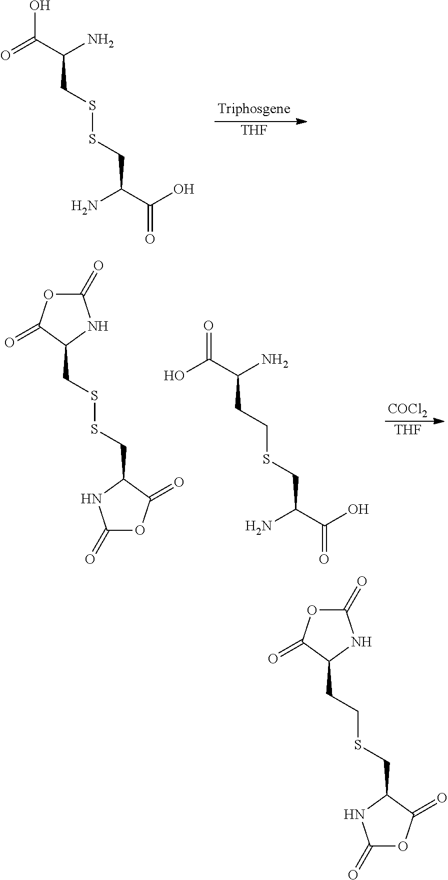

The carboxyanhydrides of cystine or cystathionine are prepared as depicted in the above scheme using triphosgene and THF.

Polypeptides and hydrogels can be prepared according the following reaction:

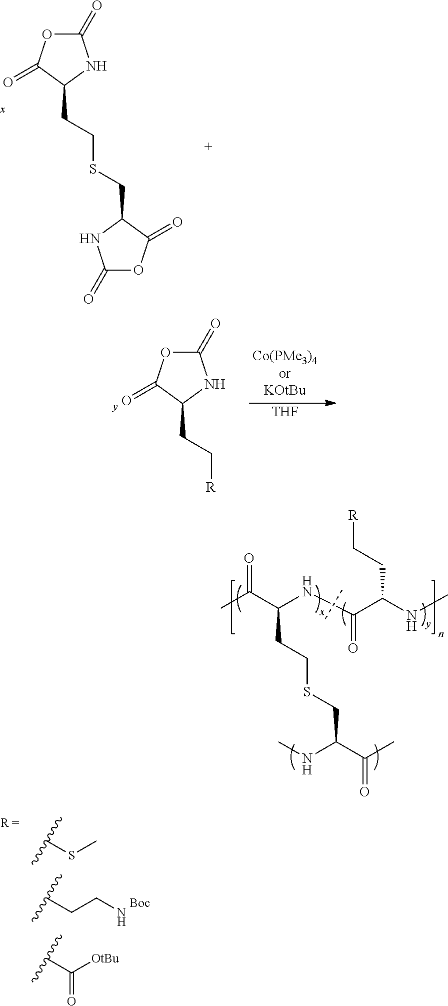

For polypeptides or hydrogels including some protected residues, the following exemplary reactions can be performed:

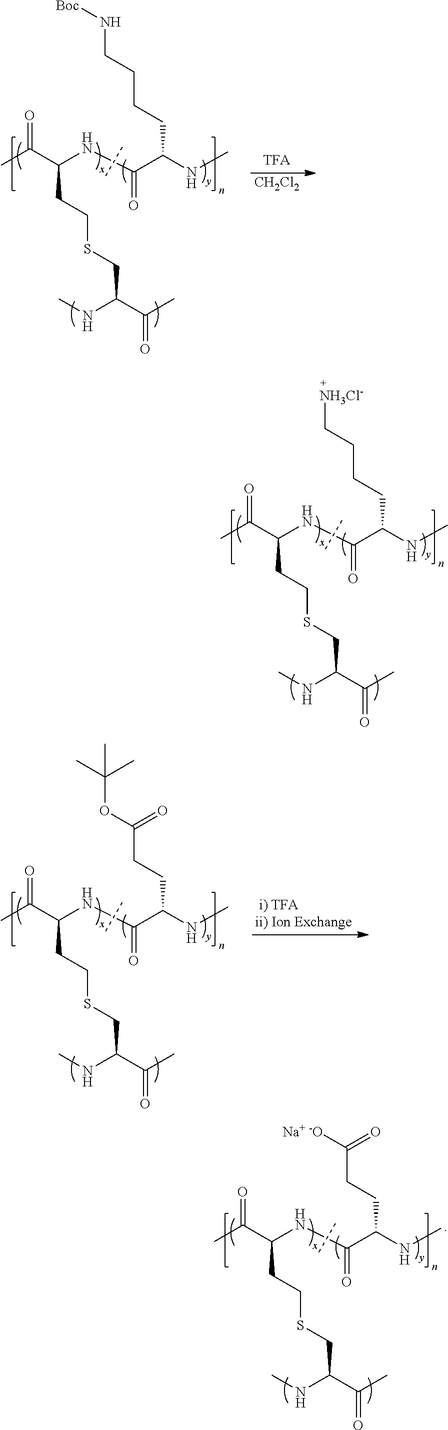

In embodiments where a hydrogel includes several crosslinkers, the reaction for manufacture can be as follows:

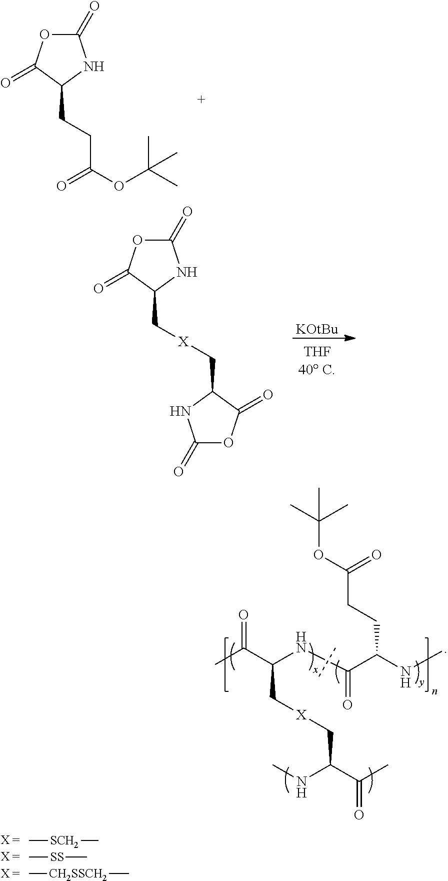

For polypeptides or hydrogels that include only one type of crosslinker, the following reaction scheme can be employed:

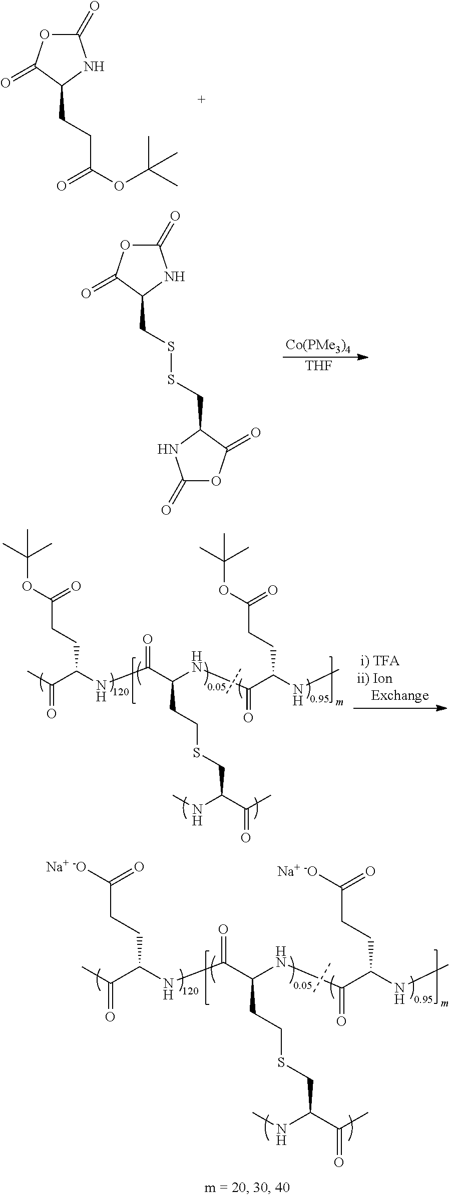

As can be seen the crosslinker can be present in a small amount relative to the remainder of the amino acid sequence. Moreover, in some embodiments, the entire polypeptide sequence can include only two amino acids, e.g. glutamate and cystathionine.

### Examples

Materials and Methods. Reactions at elevated temperature were controlled using a Corning PC 420D thermostated hotplate equipped with a thermocouple probe. Room temperature reactions were performed at ca. 22° C. ambient temperature. L-homocysteine thiolactone hydrochloride, 3-chloro-L-alanine and 2 kDa PEG-isocyanate were prepared by conventional methods. NCA purification and polymerizations were performed in an N₂ filled glove box using conventional techniques. All reactions were performed under N₂, and unless otherwise stated at 22° C. THF and hexanes were degassed by sparging with nitrogen and dried by passage through basic alumina columns. Thin-layer chromatography was performed with EMD gel 60 F254 plates (0.25 mm thickness) and visualized using a UV lamp or permanganate stain. Column chromatography was performed using Silicycle Siliaflash G60 silica (60-200 μm). H₂O was purified by reverse osmosis. Dowex 50 w×8 (Sigma-Aldrich) was washed with 1 N HCl_{(aq)}, neutralized with 1 N NH_{3(aq)} and then regenerated with 1N HCl_{(aq)} before use. TEA (Fisher) was distilled from CaH₂ under N₂ and stored over 4 Å molecular sieves. TMSCl (Sigma-Aldrich) was purified by distillation under N₂. The following chemicals were used as received from the vendor benzoyl chloride (EM Scientific), trifluoroacetic acid (Oakwood), L-selenomethionine (Chem-Impex Intl.), 2-chloroethanol (Acros), 15% phosgene in toluene (Sigma-Aldrich), L-homocystine (Chem-Impex Intl.), triphosgene (Oakwood), L-Homocysteine thiolactone hydrochloride (Sigma-Aldrich). IR spectroscopy was performed on a PerkinElmer Spectrum RX spectrometer or a JASCO FT/IR-4100 spectrometer. NMR spectroscopy was performed on a Bruker AV400 spectrometer. GPC-MALS was performed on an xStream H₂O (Jordi Labs) mixed bed column with 0.5 wt % potassium trifluoroacetate in HFiP as the eluent at 60° C. using Wyatt DAWN EOS LS and Optilab rEX RI detectors fed by a SSI Accuflow Series III pump. ESI-MS spectra were recorded on a Waters LCT Premier spectrometer. Abbreviations: Acetic acid (AcOH), attenuated total reflectance infrared spectroscopy (ATR-IR), chlorotrimethylsilane (TMSCl), circular dichroism (CD), electrospray ionization mass spectrometry (ESI-MS), 1,1,1,3,3,3-hexafluoroisopropanol (HFiP), methoxy polyethylene glycol (PEG), molar equivalent (eq), molecular weight cutoff (MWCO), triethylamine (TEA), trifluoroacetic acid (TFA), trimethylsilyl (TMS), tetrahydrofuran (THF)

L-Cystathionine

Original method: L-Homocysteine thiolactone hydrochloride (0.40 g, 2.6 mmol, 1.0 eq) and 3-chloro-L-alanine (0.32 g, 2.6 mmol, 1.0 eq) were added to a flask and flushed with N₂. Degassed 2 N NaOH (6.5 mL, 13 mmol, 5.0 eq) was added. The homogenous solution was stirred for 48 h. The mixture was concentrated in vacuo and crude ¹H NMR demonstrated a complex mixture of starting materials, L-Cystathionine, and serine. The residue was dissolved in 50 mL of water containing a few drops of 10% Na₂SO₃, and acidified to pH 1.5 with con. HCl_{(aq)}. The crude mixture was desalted on a Dowex 50 w×8 column as previously described. The recovered material was recrystallized from boiling water. The amino acid L-Cystathionine was recovered as a colorless solid (50 mg, 12% yield).

Optimized method: L-Homocysteine thiolactone hydrochloride (0.60 g, 3.9 mmol, 1.0 eq) and 3-chloro-L-alanine (0.63 g, 5.1 mmol, 1.3 eq) were added to a flask and flushed with N₂. Degassed 5 N NaOH (3.5 mL, 18 mmol, 4.5 eq) was added. The mixture was stirred 64 h. The pH was adjusted to 10.5-11.0 with con. HCl_{(aq)}. The mixture was cooled to 4° C. and allowed to stand for 16 h. The precipitate was collected by vacuum filtration. The amino acid L-Cystathionine was recovered as a colorless solid (470 mg, 54% yield). ¹H NMR (400 MHz, D₂O, 25° C.): ): 5 (4 dd, J=7.3, 4.5 Hz, 1H), 4.20 (t, J=6.5 Hz, 1H), 3.25 (dd, J=15.2, 4.6 Hz, 1H), 3.15 (dd, J=15.1, 7.2 Hz, 1H), 2.80 (tm, J=7.6 Hz, 2H), 2.30 (m, 1H), 2.21 (m, 1H).

L-Cystathionine bis-NCA

L-Cystathionine (0.30 g, 1.3 mmol, 1.0 eq) was suspended in THF (20 mL). 15% phosgene solution (3.9 mL, 5.4 mmol, 4 eq) was added. The mixture was stirred at 45° C. for 18 h and then concentrated. The crude product was purified by column chromatography (60% THF/Hexanes) under inert atmosphere. After concentration, the material was diluted with THF and precipitated into hexanes. This provided L-Cystathionine bis-NCA (0.20 g, 54% yield) as a colorless solid. ¹H NMR (400 MHz, CD₃CN, 25° C.): 6.86 (br m, 2H), 4.62 (dd, J=4.2, 1.3 Hz, 1H), 4.44 (dd, J=1.4, 5.6 Hz, 1H), 3.04 (dd, J=14.5, 4.0 Hz, 1H), 2.92 (dd, J=14.5, 5.2 Hz, 1H), 2.71 (t, J=7.4 Hz, 2H), 2.06 (m, 2H). ¹³C NMR (100 MHz, CD₃CN, 25° C.): 170.9, 169.7, 151.8, 151.8, 58.2, 56.1, 32.4, 30.8, 28.0. IR (thin film) 1860, 1792 cm⁻¹. ESI-MS m/z=273.0204 [M-H]⁻ (calcd 273.0181 for C₉H₉N₂O₆S₂).

L-Cystine Bis-NCA

L-cystine (1.5 g, 6.2 mmol, 1.0 eq) was suspended in THF (50 mL). Triphosgene (2.5 g, 8.3 mmol, 1.3 eq) was added in one portion. The mixture was stirred at 50° C. for 24 h. The turbid mixture was concentrated and the crude product was purified by column chromatography (50% THF/Hexanes) under inert atmosphere. After concentration, the material was diluted with THF and precipitated into hexanes. This L-Cystine Bis-NCA (0.42 g, 23% yield) as a pale yellow solid.

L-Homocystine Bis-NCA

L-Homocystine (0.4 g, 1.5 mmol, 1.0 eq) was suspended in THF (30 mL). triphosgene (0.59 g, 2.0 mmol, 1.3 eq) was added in one portion. The mixture was stirred at 50° C. for 24 h. The turbid mixture was concentrated and the crude product was purified by column chromatography (60% THF/Hexanes) under inert atmosphere. After concentration, the material was diluted with THF and precipitated into hexanes. This provided L-Homocystine Bis-NCA (0.20 g, 42% yield) as a colorless solid. ¹H NMR (400 MHz, CD₃CN, 25° C.): 6.87 (br s, 2H), 4.47 (ddd, J=6.9, 5.4, 1.3 Hz, 2H), 2.80 (t, J=7.2 Hz, 4H), 2.19 (m, 4H). ¹³C NMR (100 MHz, CD₃CN, 25° C.): 172.1, 153.2, 57.5, 34.2, 31.8. IR (thin film) 1860, 1792 cm⁻¹. ESI-MS m/z=319.0032 [M-H]⁻ (calcd 319.0059 for C₁₀H₁₁N₂O₆S₂).

L-Cystathionine Copolymers, Co(PMe₃)₄ Initiated)

Equally concentrated solutions of L-cystathionine bis-NCA and co-monomers. Solutions of methionine NCA (0.18 M), t-butoxycarbonyl protected lysine NCA (0.18 M) or t-butyl protected glutamic acid (0.18 M) were prepared in THF. Two of these solutions were mixed in proportions for the desired cystathionine bis-NCA/comonomer feed ratio. The resultant solutions were treated with 50 mM Co(PMe₃)₄ in THF, at the requisite monomer to initiator ratio. The progress of the polymerizations was monitored by IR spectroscopy. After 16 h the polymerizations were removed from the glovebox and stirred with an excess of water to precipitate the polymers. The mixtures were centrifuged and the supernatant was discarded. The precipitate was once again triturated with water and isolated in a similar manner. The resultant precipitate was freed of volatiles in vacuo.

Bis-NCA Copolymers, KOtBu Initiated

Stock solutions of bis-NCA monomers of cystathionine, cystine, and homocystine and mono-NCA monomers of t-butoxycarboxyl protected lysine and t-butyl protected glutamic acid were prepared in THF at a concentration of 0.18 M. These were mixed in proportions to provide the desired comonomer feed ratio. The resultant solutions were treated with the initiator, 50 mM KOtBu in THF, at the requisite monomer to initiator ratio. The mixtures were heated at 40° C. for 24 h. An aliquot of the polymerization mixture was analyzed by IR spectroscopy to confirm polymerization completion. The polymerizations were concentrated under a gentle stream of air followed by high vacuum. The obtained solids were deprotected without further purification.

Diblock Copolymers Containing L-Cystathionine

First Block: A 0.18 M solution of 5-tert-butyl L-glutamate NCA in THF was treated with 50 mM Co(PMe₃)₄ at a 60:1 monomer to initiator ratio. The polymerization was allowed to proceed for 2 h, at which point full consumption of monomer was confirmed by IR spectroscopy of a reaction mixture aliquot. Second Block: A comonomer stock solution (0.18 M total monomer concentration) containing a 5:95 ratio of cystathionine-NCA and 5-tert-butyl L-glutamate NCA was prepared. Three aliquots were withdrawn from the polymerization mixture and these were treated with the comonomer solution at 10, 20 and 30:1 ratios, relative to the concentration of cobalt initiator in each aliquot. The polymerizations were allowed to stand for 16 h, at which point full monomer consumption was determined by IR spectroscopy. The polymerization mixtures were concentrated under a stream of air and then triturated sequentially with dilute AcOH (1×) followed by water (3×). The resultant wet solid was concentrated by lyophilization.

Ionic Hydrogels

Protected copolymer was treated with TFA (40 μL/mg of polymer) and allowed to stand for 24 h. The mixture was then evaporated under a stream of air to provide a solvent-swollen solid. Volatiles were completely removed under high vacuum. The crude polymer was transferred to a 2 kDa MWCO dialysis bag and dialyzed against aqueous 50 mM NaCl (8 h, 2 solvent changes) followed by H₂O (16 h, 3 solvent changes). The retentate was lyophilized to provide the deprotected copolymer. A list of various polypeptides is summarized in Table 1.

TABLE 1
Examples of amino acid combinations used to synthesize stable
and stimuli-responsive polypeptide materials, their mixtures,
their gels, and their hydrogels

| | | Disulfide | | |
|---|---|---|---|---|
| Poly- | Monosulfide | amino | Amino | Class of |
| peptide | Amino Acid | acid | acid | polypeptide |
| 1 | Cystathionine | | Alanine | stable synthetic |
| | | | | polypeptide |
| | | | | network material |
| 2 | Cystathionine | | Arginine | stable synthetic |
| | | | | polypeptide |
| | | | | network material |
| 3 | Cystathionine | | Asparagine | stable synthetic |
| | | | | polypeptide |
| | | | | network material |
| 4 | Cystathionine | | Aspartic | stable synthetic |
| | | | acid | polypeptide |
| | | | | network material |
| 5 | Cystathionine | | Cysteine | stable synthetic |
| | | | | polypeptide |
| | | | | network material |
| 6 | Cystathionine | | Glutamine | stable synthetic |
| | | | | polypeptide |
| | | | | network material |
| 7 | Cystathionine | | Glutamic | stable synthetic |
| | | | acid | polypeptide |
| | | | | network material |
| 8 | Cystathionine | | Glycine | stable synthetic |
| | | | | polypeptide |
| | | | | network material |
| 9 | Cystathionine | | Histidine | stable synthetic |
| | | | | polypeptide |
| | | | | network material |
| 10 | Cystathionine | | Isoleucine | stable synthetic |
| | | | | polypeptide |
| | | | | network material |
| 11 | Cystathionine | | Leucine | stable synthetic |
| | | | | polypeptide |
| | | | | network material |
| 12 | Cystathionine | | Lysine | stable synthetic |
| | | | | polypeptide |
| | | | | network material |
| 13 | Cystathionine | | Methionine | stable synthetic |
| | | | | polypeptide |
| | | | | network material |
| 14 | Cystathionine | | Phenyala- | stable synthetic |
| | | | nine | polypeptide |
| | | | | network material |
| 15 | Cystathionine | | Proline | stable synthetic |
| | | | | polypeptide |
| | | | | network material |
| 16 | Cystathionine | | Serine | stable synthetic |
| | | | | polypeptide |
| | | | | network material |
| 17 | Cystathionine | | Threonine | stable synthetic |
| | | | | polypeptide |
| | | | | network material |
| 18 | Cystathionine | | Tryptophan | stable synthetic |
| | | | | polypeptide |
| | | | | network material |
| 19 | Cystathionine | | Tyrosine | stable synthetic |
| | | | | polypeptide |
| | | | | network material |
| 20 | Cystathionine | | Valine | stable synthetic |
| | | | | polypeptide |
| | | | | network material |
| 21 | | Cystine | Glutamic | stimuli-responsive |
| | | | acid | synthetic |
| | | | | polypeptide |
| | | | | network material |
| 22 | | Homo- | Glutamic | stimuli-responsive |
| | | cystine | acid | synthetic |
| | | | | polypeptide |
| | | | | network material |
| 23 | Cystathionine | Cystine | Alanine | stimuli-responsive |
| | | | | synthetic |
| | | | | polypeptide |
| | | | | network material |
| 24 | Cystathionine | Cystine | Cysteine | stimuli-responsive |
| | | | | synthetic |
| | | | | polypeptide |
| | | | | network material |
| 25 | Cystathionine | Cystine | Glutamic | stimuli-responsive |
| | | | acid | synthetic |
| | | | | polypeptide |
| | | | | network material |
| 26 | Cystathionine | Cystine | Histidine | stimuli-responsive |
| | | | | synthetic |
| | | | | polypeptide |
| | | | | network material |
| 27 | Cystathionine | Cystine | Lysine | stimuli-responsive |
| | | | | synthetic |
| | | | | polypeptide |
| | | | | network material |
| 28 | Cystathionine | Cystine | Methionine | stimuli-responsive |
| | | | | synthetic |
| | | | | polypeptide |
| | | | | network material |
| 29 | Cystathionine | Cystine | Proline | stimuli-responsive |
| | | | | synthetic |
| | | | | polypeptide |
| | | | | network material |
| 30 | Cystathionine | Cystine | Tyrosine | stimuli-responsive |
| | | | | synthetic |
| | | | | polypeptide |
| | | | | network material |
| 31 | Cystathionine | Homo- | Alanine | stimuli-responsive |
| | | cystine | | synthetic |
| | | | | polypeptide |
| | | | | network material |
| 32 | Cystathionine | Homo- | Cysteine | stimuli-responsive |
| | | cystine | | synthetic |
| | | | | polypeptide |
| | | | | network material |
| 33 | Cystathionine | Homo- | Glutamic | stimuli-responsive |
| | | cystine | acid | synthetic |
| | | | | polypeptide |
| | | | | network material |
| 34 | Cystathionine | Homo- | Histidine | stimuli-responsive |
| | | cystine | | synthetic |
| | | | | polypeptide |
| | | | | network material |
| 35 | Cystathionine | Homo- | Lysine | stimuli-responsive |
| | | cystine | | synthetic |
| | | | | polypeptide |
| | | | | network material |
| 36 | Cystathionine | Homo- | Methionine | stimuli-responsive |
| | | cystine | | synthetic |
| | | | | polypeptide |
| | | | | network material |
| 37 | Cystathionine | Homo- | Proline | stimuli-responsive |
| | | cystine | | synthetic |
| | | | | polypeptide |
| | | | | network material |
| 38 | Cystathionine | Homo- | Tyrosine | stimuli-responsive |
| | | cystine | | synthetic |
| | | | | polypeptide |
| | | | | network material |
| 39 | Cystathionine | Cystine, | Alanine | stimuli-responsive |
| | | Homo- | | synthetic |
| | | cystine | | polypeptide |
| | | | | network material |
| 40 | Cystathionine | Cystine, | Cysteine | stimuli-responsive |
| | | Homo- | | synthetic |
| | | cystine | | polypeptide |
| | | | | network material |
| 41 | Cystathionine | Cystine, | Glutamic | stimuli-responsive |
| | | Homo- | acid | synthetic |
| | | cystine | | polypeptide |
| | | | | network material |
| 42 | Cystathionine | Cystine, | Histidine | stimuli-responsive |
| | | Homo- | | synthetic |
| | | cystine | | polypeptide |
| | | | | network material |
| 43 | Cystathionine | Cystine, | Lysine | stimuli-responsive |
| | | Homo- | | synthetic |
| | | cystine | | polypeptide |
| | | | | network material |
| 44 | Cystathionine | Cystine, | Methionine | stimuli-responsive |
| | | Homo- | | synthetic |
| | | cystine | | polypeptide |
| | | | | network material |
| 45 | Cystathionine | Cystine, | Proline | stimuli-responsive |
| | | Homo- | | synthetic |
| | | cystine | | polypeptide |
| | | | | network material |
| 46 | Cystathionine | Cystine, | Tyrosine | stimuli-responsive |
| | | Homo- | | synthetic |
| | | cystine | | polypeptide |
| | | | | network material |
| 47 | Lanthionine | | Cysteine | stable synthetic |
| | | | | polypeptide |
| | | | | network material |
| 48 | Lanthionine | | Glutamic | stable synthetic |
| | | | acid | polypeptide |
| | | | | network material |
| 49 | Lanthionine | | Lysine | stable synthetic |
| | | | | polypeptide |
| | | | | network material |
| 50 | Lanthionine | | Methionine | stable synthetic |
| | | | | polypeptide |
| | | | | network material |
| 51 | Lanthionine | | Tyrosine | stable synthetic |
| | | | | polypeptide |
| | | | | network material |
| 52 | Lanthionine | Cystine | Cysteine | stimuli-responsive |
| | | | | synthetic |
| | | | | polypeptide |
| | | | | network material |
| 53 | Lanthionine | Cystine | Glutamic | stimuli-responsive |
| | | | acid | synthetic |
| | | | | polypeptide |
| | | | | network material |
| 54 | Lanthionine | Cystine | Lysine | stimuli-responsive |
| | | | | synthetic |
| | | | | polypeptide |
| | | | | network material |
| 55 | Lanthionine | Cystine | Methionine | stimuli-responsive |
| | | | | synthetic |
| | | | | polypeptide |
| | | | | network material |
| 56 | Lanthionine | Cystine | Tyrosine | stimuli-responsive |
| | | | | synthetic |
| | | | | polypeptide |
| | | | | network material |
| 57 | Lanthionine | Cystine, | Cysteine | stimuli-responsive |
| | | Homo- | | synthetic |
| | | cystine | | polypeptide |
| | | | | network material |
| 58 | Lanthionine | Cystine, | Glutamic | stimuli-responsive |
| | | Homo- | acid | synthetic |
| | | cystine | | polypeptide |
| | | | | network material |
| 59 | Lanthionine | Cystine, | Lysine | stimuli-responsive |
| | | Homo- | | synthetic |
| | | cystine | | polypeptide |
| | | | | network material |
| 60 | Lanthionine | Cystine, | Methionine | stimuli-responsive |
| | | Homo- | | synthetic |
| | | cystine | | polypeptide |
| | | | | network material |
| 61 | Lanthionine | Cystine, | Tyrosine | stimuli-responsive |
| | | Homo- | | synthetic |
| | | cystine | | polypeptide |
| | | | | network material |
| 62 | Homolanthionine | | Cysteine | stable synthetic |
| | | | | polypeptide |
| | | | | network material |
| 63 | Homolanthionine | | Glutamic | stable synthetic |
| | | | acid | polypeptide |
| | | | | network material |
| 64 | Homolanthionine | | Lysine | stable synthetic |
| | | | | polypeptide |
| | | | | network material |
| 65 | Homolanthionine | | Methionine | stable synthetic |
| | | | | polypeptide |
| | | | | network material |
| 66 | Homolanthionine | | Tyrosine | stable synthetic |
| | | | | polypeptide |
| | | | | network material |
| 67 | Homolanthionine | Cystine | Cysteine | stimuli-responsive |
| | | | | polypeptide |
| | | | | network material |
| 68 | Homolanthionine | Cystine | Glutamic | stimuli-responsive |
| | | | acid | polypeptide |
| | | | | network material |
| 69 | Homolanthionine | Cystine | Lysine | stimuli-responsive |
| | | | | polypeptide |
| | | | | network material |
| 70 | Homolanthionine | Cystine | Methionine | stimuli-responsive |
| | | | | polypeptide |
| | | | | network material |
| 71 | Homolanthionine | Cystine | Tyrosine | stimuli-responsive |
| | | | | polypeptide |
| | | | | network material |
| 72 | Homolanthionine | Cystine, | Cysteine | stimuli-responsive |
| | | Homo- | | polypeptide |
| | | cystine | | network material |
| 73 | Homolanthionine | Cystine, | Glutamic | stimuli-responsive |
| | | Homo- | acid | polypeptide |
| | | cystine | | network material |
| 74 | Homolanthionine | Cystine, | Lysine | stimuli-responsive |
| | | Homo- | | polypeptide |
| | | cystine | | network material |
| 75 | Homolanthionine | Cystine, | Methionine | stimuli-responsive |
| | | Homo- | | polypeptide |
| | | cystine | | network material |
| 76 | Homolanthionine | Cystine, | Tyrosine | stimuli-responsive |
| | | Homo- | | polypeptide |
| | | cystine | | network material |
| 77 | Cystathionine, | | Cysteine | stable synthetic |
| | Lanthionine | | | polypeptide |
| | | | | network material |
| 78 | Cystathionine, | | Glutamic | stable synthetic |
| | Lanthionine | | acid | polypeptide |
| | | | | network material |
| 79 | Cystathionine, | | Lysine | stable synthetic |
| | Lanthionine | | | polypeptide |
| | | | | network material |
| 80 | Cystathionine, | | Methionine | stable synthetic |
| | Lanthionine | | | polypeptide |
| | | | | network material |
| 81 | Cystathionine, | | Tyrosine | stable synthetic |
| | Lanthionine | | | polypeptide |
| | | | | network material |
| 82 | Cystathionine, | Cystine | Cysteine | stimuli-responsive |
| | Lanthionine | | | polypeptide |
| | | | | network material |
| 83 | Cystathionine, | Cystine | Glutamic | stimuli-responsive |
| | Lanthionine | | acid | polypeptide |
| | | | | network material |
| 84 | Cystathionine, | Cystine | Lysine | stimuli-responsive |
| | Lanthionine | | | polypeptide |
| | | | | network material |
| 85 | Cystathionine, | Cystine | Methionine | stimuli-responsive |
| | Lanthionine | | | polypeptide |
| | | | | network material |
| 86 | Cystathionine, | Cystine | Tyrosine | stimuli-responsive |
| | Lanthionine | | | polypeptide |
| | | | | network material |
| 87 | Cystathionine, | Homo- | Cysteine | stimuli-responsive |
| | Lanthionine | cystine | | polypeptide |
| | | | | network material |
| 88 | Cystathionine, | Homo- | Glutamic | stimuli-responsive |
| | Lanthionine | cystine | acid | polypeptide |
| | | | | network material |
| 89 | Cystathionine, | Homo- | Lysine | stimuli-responsive |
| | Lanthionine | cystine | | polypeptide |
| | | | | network material |
| 90 | Cystathionine, | Homo- | Methionine | stimuli-responsive |
| | Lanthionine | cystine | | polypeptide |
| | | | | network material |
| 91 | Cystathionine, | Homo- | Tyrosine | stimuli-responsive |
| | Lanthionine | cystine | | polypeptide |
| | | | | network material |
| 92 | Cystationine, | Cystine, | Cysteine | stimuli-responsive |
| | Lanthionine, | Homo- | | polypeptide |
| | Homolanthionine | cystine | | network material |
| 93 | Cystathionine, | Cystine, | Glutamic | stimuli-responsive |
| | Lanthionine, | Homo- | acid | polypeptide |
| | Homolanthionine | cystine | | network material |
| 94 | Cystathionine, | Cystine, | Lysine | stimuli-responsive |
| | Lanthionine, | Homo- | | polypeptide |
| | Homolanthionine | cystine | | network material |
| 95 | Cystathionine, | Cystine, | Methionine | stimuli-responsive |
| | Lanthionine, | Homo- | | polypeptide |
| | Homolanthionine | cystine | | network material |
| 96 | Cystathionine, | Cystine, | Tyrosine | stimuli-responsive |
| | Lanthionine, | Homo- | | polypeptide |
| | Homolanthionine | cystine | | network material |

As used herein, the terms “comprises,” “comprising,” “includes,” “including” “has,” “having” or any other variation thereof, are intended to cover a non-exclusive inclusion. For example, a process method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and Bis true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

In the foregoing specification, the concepts have been described with reference to specific embodiments. However, one of ordinary skill in the art appreciates that various modifications and changes can be made without departing from the scope of the invention as set forth in the claims below. Accordingly, the specification are to be regarded in an illustrative rather than a restrictive sense, and all such modifications are intended to be included within the scope of invention.

1. A polypeptide comprising amino acids in an amino acid sequence, wherein a portion of at least 0.001%, at least 0.005%, at least 0.01%, at least 0.03%, at least 0.05%, at least 0.07%, at least 0.09%, at least 0.1%, at least 0.15%, at least 0.2%, at least 0.25%, at least 0.3%, at least 0.35%, at least 0.4%, at least 0.45%, at least 0.5%, or at least 0.6% of the amino acids are covalently linked via a monothioether bridge.

2. The polypeptide according to claim 1, wherein the portion includes amino acids selected from 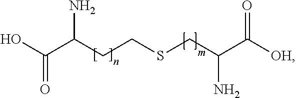
wherein n is 0, 1, or 2 and m is 1 or 2.

3. The polypeptide according to claim 1, wherein the portion includes amino acids selected from

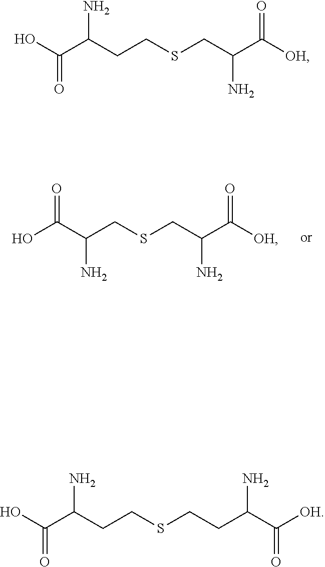 [SMILES: NC(CCSCC(N)C(=O)O)C(=O)O ; NC(CSCC(N)C(=O)O)C(=O)O ; "or" ; NC(CCSCCC(N)C(=O)O)C(=O)O]

4. The polypeptide according to claim 3, wherein the portion includes amino acids selected from

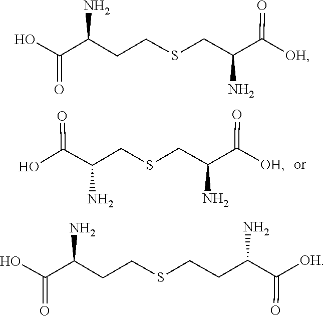

5. The polypeptide according to claim 1, wherein the amino acids not included in the portion are selected from a subset, wherein the subset includes not more than 10 amino acids, not more than 9 amino acids, not more than 8 amino acids, not more than 7 amino acids, not more than 6 amino acids, not more than 5 amino acids, not more than 4 amino acids, not more than 3 amino acids, or not more than 2 amino acids selected from the group consisting of alanine, arginine, asparagine, aspartic acid, cysteine, homocysteine, 2,4-diaminobutyric acid, glutamic acid, homoglutamic acid, glutamine, homoglutamine, glycine, histidine, isoleucine, leucine, lysine, methionine, phenylalanine, ornithine, proline, serine, homoserine, threonine, tryptophan, tyrosine, and valine.

6. The polypeptide according to claim 1, wherein the polypeptide further includes a cysteine portion, the cysteine portion being at least 0.01%, at least 0.02%, at least 0.04%, at least 0.06%, at least 0.08%, at least 0.1%, at least 0.15%, at least 0.2%, at least 0.25%, at least 0.3%, at least 0.35%, at least 0.4%, at least 0.45%, at least 0.5%, at least 0.55%, at least 0.6%, at least 0.65%, at least 0.7%, at least 0.75%, or at least 0.8%.

7. The polypeptide according to claim 6, wherein a ratio of the portion to the cysteine portion is not greater than 1, not greater than 0.95, not greater than 0.9, not greater than 0.85, not greater than 0.8, not greater than 0.75, not greater than 0.7, not greater than 0.65, not greater than 0.6, not greater than 0.55, not greater than 0.5, or not greater than 0.45.

8. The polypeptide according to claim 6, wherein a ratio of the portion to the cysteine portion is greater than 1, greater than 1.1, greater than 1.2, greater than 1.3, greater than 1.4, greater than 1.5, greater than 1.6, greater than 1.8, greater than 2, greater than 2.4, greater than 2.8, greater than 3.2, greater than 3.6, greater than 4, greater than 4.5, greater than 5, greater than 5.5, greater than 6, greater than 7, greater than 8, greater than 10, or greater than 15.

9. The polypeptide according to claim 1, wherein the portion is not greater than 60%, not greater than 50%, not greater than 40%, not greater than 30%, not greater than 25%, not greater than 20%, not greater than 15%, not greater than 10%, not greater than 9%, not greater than 8%, not greater than 7.5%, not greater than 7%, not greater than 6.5%, not greater than 6%, not greater than 5.5%, not greater than 5%, not greater than 4.8%, not greater than 4.6%, not greater than 4.4%, not greater than 4.2%, not greater than 4%, not greater than 3.8%, not greater than 3.6%, not greater than 3.4%, not greater than 3.2%, not greater than 3%, not greater than 2.8%, not greater than 2.6%, not greater than 2.4%, not greater than 2.2%, not greater than 2%, not greater than 1.8%, or not greater than 1.6% of the amino acid sequence.

10. The polypeptide according to claim 1 comprising the structure: 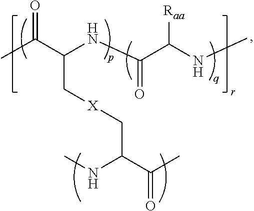
wherein X for each occurrence is independently selected from S, S—CH₂, CH₂—S, S—S, CH₂—S—S—CH₂, or CH₂—S—CH₂, Rₐₐ is an amino acid residue, p and q are integers independently selected from 1 through 50, p/q≤1, and r is an integer not less than 20, not less than 30, not less than 40.

11. The polypeptide according to claim 10, selected from the group consisting of:
(i) X for each occurrence is independently selected from S, S—CH₂, CH₂—S, or CH₂—S—CH₂;
(ii) Rₐₐ is selected from —CH₂COOH or a salt thereof, —CH₂CH₂COOH or a salt thereof, —CH₂CH₂CH₂CH₂NH₂ or a salt thereof, CH₂OH, CH₂(CH₃)OH, or —CH₂CH₂SCH₃;
(iii) p/q is ≤0.9, ≤0.8, ≤0.7, ≤0.6, ≤0.5, ≤0.4, ≤0.3, ≤0.25, ≤0.2, ≤0.15, ≤0.1, ≤0.08, ≤0.06, ≤0.05, ≤0.04, ≤0.03, or ≤0.02; and
(iv) any combination of (i), (ii), and (iii).

12. A hydrogel comprising a polymer having a crosslinker comprising a structure 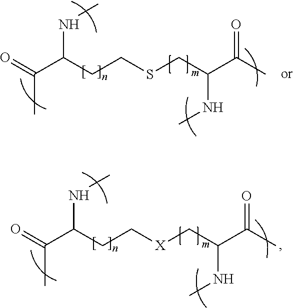
wherein n is 0, 1, or 2 and m is 1 or 2, and X is selected from a single bond, CH₂, CH(OH), C(O), NH, O, S(O), S(O)₂, or Se;
wherein the hydrogel has at least one property selected from the group consisting of:
(i) the hydrogel maintains gelation in the presence of a reduction agent selected from a mercapto compound or dithiothreitol;
(ii) the hydrogel is an element of a pharmaceutical formulation adapted to release an active ingredient;
(iii) the hydrogel is an element of an engineered tissue for biological tissue replacement;
(iv) the hydrogel is adapted to absorb pollutants from a liquid;
(v) the hydrogel is an element of an electrode adapted to measure one or more carbohydrates present in organic and biological fluids and tissues;
(vi) the hydrogel is selfhealing;
(vii) the hydrogel has a peel strength according to ASTM F2256 (2015) of at least 0.1 N, at least 0.2 N, at least 0.4 N, at least 0.6 N, at least 0.8 N, at least 1.0 N, at least 1.5 N, at least 2 N, at least 4 N, at least 6 N, at least 8 N, or at least 10 N; and
(viii) any combination of (i), (ii), (iii), (iv), (v), (vi), and (vii).

13. The hydrogel according to claim 12, further comprising the structure: 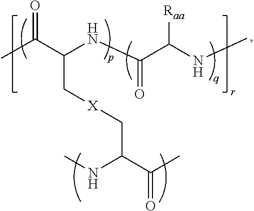
wherein X for each occurrence is independently selected from —S—, S—CH₂—, —CH₂—S, or —CH₂—S—CH₂—, Rₐₐ is an amino acid residue, p and q are integers independently selected from 1 through 50, and r is an integer.

14. The hydrogel according to claim 13, wherein p/q≤1, and r is an integer not less than 20, not less than 30, not less than 40.

15. The hydrogel according to claim 12, wherein the polymer further includes a section selected from the group consisting of a polypeptide, a polysaccharide, a polyethylene glycol, a poly vinyl alcohol, a polyacrylate, a polyurethane, a polyamine, and any combination thereof.

16. A polypeptide comprising amino acids in an amino acid sequence and a portion of the amino acid sequence includes an amino acid selected from 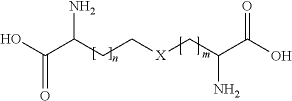
wherein n is 0, 1, or 2 and m is 1 or 2, and X is selected from a single bond, CH₂, CH(OH), C(O), NH, O, S(O), S(O)₂, or Se.

17. The polypeptide according to claim 16, wherein the portion is at least 0.001%, at least 0.005%, at least 0.01%, at least 0.03%, at least 0.05%, at least 0.07%, at least 0.09%, at least 0.1%, at least 0.15%, at least 0.2%, at least 0.25%, at least 0.3%, at least 0.35%, at least 0.4%, at least 0.45%, at least 0.5%, or at least 0.6% of the amino acid sequence.

18. The polypeptide according to claim 16, wherein the portion is not greater than 95%, not greater than 90%, not greater than 85%, not greater than 80%, not greater than 75%, not greater than 70%, not greater than 65%, not greater than 60%, not greater than 55%, not greater than 50%, not greater than 45%, not greater than 40%, not greater than 35%, not greater than 30%, not greater than 28%, not greater than 26%, not greater than 24%, not greater than 22%, not greater than 20%, not greater than 18%, not greater than 16%, not greater than 14%, not greater than 12%, not greater than 10%, not greater than 9%, not greater than 8%, not greater than 7%, not greater than 6%, not greater than 5%, not greater than 4%, not greater than 3%, or not greater than 2% of the amino acid sequence.

19. The polypeptide according to claim 16, wherein the amino acids not included in the portion are selected from a subset, wherein the subset includes not more than 10 amino acids, not more than 9 amino acids, not more than 8 amino acids, not more than 7 amino acids, not more than 6 amino acids, not more than 5 amino acids, not more than 4 amino acids, not more than 3 amino acids, or not more than 2 amino acids selected from the group consisting of alanine, arginine, asparagine, aspartic acid, cysteine, homocysteine, 2,4-diaminobutyric acid, glutamic acid, homoglutamic acid, glutamine, homoglutamine, glycine, histidine, isoleucine, leucine, lysine, methionine, phenylalanine, ornithine, proline, serine, homoserine, threonine, tryptophan, tyrosine, and valine.

20. The polypeptide according to claim 16, wherein a section of amino acids that is not included in the portion is not greater than 99%, not greater than 98%, not greater than 95%, not greater than 90%, not greater than 80%, not greater than 70%, not greater than 60%, not greater than 50%, not greater than 40%, not greater than 30%, not greater than 25%, not greater than 20%, not greater than 15%, not greater than 10%, not greater than 9%, not greater than 8%, not greater than 7.5%, not greater than 7%, not greater than 6.5%, not greater than 6%, not greater than 5.5%, not greater than 5%, not greater than 4.8%, not greater than 4.6%, not greater than 4.4%, not greater than 4.2%, not greater than 4%, not greater than 3.8%, not greater than 3.6%, not greater than 3.4%, not greater than 3.2%, not greater than 3%, not greater than 2.8%, not greater than 2.6%, not greater than 2.4%, not greater than 2.2%, not greater than 2%, not greater than 1.8%, or not greater than 1.6% of the amino acid sequence.

21. (canceled)

22. (canceled)
